(19) United States
(12) Patent Application Publication
(10) Pub. No.: US 2023/0223115 A1
(43) Pub. Date: Jul. 13, 2023

(54) RECORDING MEDIUM WHERE MEDICAL QUESTIONNAIRE SUPPORT PROGRAM IS RECORDED, AND MEDICAL QUESTIONNAIRE SUPPORT SYSTEM

(71) Applicant: SayWow Co., Ltd., Miyazaki (JP)

(72) Inventor: Hisaharu NISHIMOTO, Miyazaki (JP)

(21) Appl. No.: 17/690,692

(22) Filed: Mar. 9, 2022

(30) Foreign Application Priority Data
Jan. 7, 2022 (JP) 2022-001939

(51) Int. Cl. G16H 10/20

(52) U.S. Cl. CPC G16H 10/20

(57) ABSTRACT

There is provided a recording medium where a medical questionnaire support program is recorded, the medical questionnaire support program causing an electronic device to execute: a symptom time checking step of checking the time of onset of a problem; a problem checking step of checking an item related to the problem in a clinical department; and a printing step of printing answer results of the steps on printing paper to be affixed.

### CROSS-REFERENCE TO RELATED APPLICATION

This application claims priority from Japanese Patent Application No. 2022-001939 filed with the Japan Patent Office on Jan. 7, 2022, the entire content of which is hereby incorporated by reference.

### BACKGROUND

### 1. Technical Field

An aspect of the present disclosure relates to a recording medium where a medical questionnaire support program is recorded, and a medical questionnaire support system.

### 2. Related Art

In the medical field, a technology for automating patient history taking by use of an electronic device such as a personal computer or a tablet is widespread (refer to JP-A-2020-194202 and JP-A-2021-149453).

In other words, in the technology, a patient answers questions on an electronic device before seeing a doctor or nurse to take a history from the patient, and the results of history taking are shared by use of a server.

### SUMMARY

There is provided a recording medium where a medical questionnaire support program is recorded, the medical questionnaire support program causing an electronic device to execute: a symptom time checking step of checking the time of onset of a problem; a problem checking step of checking an item related to the problem in a clinical department; and a printing step of printing answer results of the steps on printing paper to be affixed.

### BRIEF DESCRIPTION OF DRAWINGS

FIG. 1 illustrates an example of a flowchart of a medical questionnaire support program (for first visit);

FIG. 2 illustrates an example of a flowchart of a medical questionnaire support program (for re-visit);

FIG. 3 illustrates examples of items of the medical questionnaire support program (for first visit);

FIG. 4 illustrates examples of items of the medical questionnaire support program (for first visit);

FIG. 5 illustrates examples of items of the medical questionnaire support program (for first visit);

FIG. 6 illustrates examples of items of the medical questionnaire support program (for re-visit);

FIGS. 7A and 7B illustrate examples of screens of the medical questionnaire support program;

FIGS. 8A to 8O illustrate examples of screens (for first visit) of the medical questionnaire support program;

FIGS. 9A to 9G illustrate examples of screens (for re-visit) of the medical questionnaire support program; and

FIG. 10 illustrates an example of a medical questionnaire support system.

### DETAILED DESCRIPTION

In the following detailed description, for purpose of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

The prior art is useful in the respect that the efficiency of history taking can be enhanced, and in the respect that information can be stored and shared as digital data. In addition, such an art can promote a dramatic improvement in convenience by sharing information including image data obtained by, for example, CT and MRI, and electronic medical charts.

However, in terms of such a system to share, for example, electronic medical charts, the introduction cost is high, and the maintenance cost is also incurred. Hence, the sharing system has been introduced into large-scale hospitals, but has not been introduced into many other hospitals. Moreover, a system to which medical image data is added is not merely a medical paperwork support system but is categorized as a medical device. Hence, medical approval is required for the use of the system.

The inventor as an ophthalmologist has been wishing to improve such circumstances.

In other words, approximately 80% of hospitals in Japan are small- or medium-sized hospitals. In these hospitals, under present circumstances, not electronic charts but paper charts are used. Digitization is progressing as the technical trend of the medical system, but in many hospitals, it cannot be said that such a digital technology is a technology where the performance is always worth the cost in the respects of business scale and cost, and therefore the digital technology is a technology that is decided not to be introduced.

In addition, as the digitization or sharing of information progresses further and further, a risk and a cost in relation to information security increase.

In other words, medical information is very important personal information and therefore is controlled to prevent the leakage of the information. Moreover, medical information is digitized in the currently dominant medical system in many cases. Therefore, it is required to increase the security of digitized medical information and to upgrade the versions of software and hardware that handle medical information at regular intervals. Hence, in the currently dominant medical system, not only the cost of introduction but also the cost to maintain the system, that is, the ongoing cost are incurred.

Considering these circumstances, the advantage of using paper charts is significant in many hospitals. Hence, the inventor considers that it is desirable to have an easy-to-use medical support system even when paper charts are used.

With the above circumstances as a backdrop, one object of the present disclosure is to provide a medical support system based on the precondition that paper medical charts are used.

As a result of a diligent study, the inventor has conceived a questionnaire system that systematizes history taking that is useful for a hospital and prints the results of history taking on sticker paper to be affixed, and has completed the technology of the present disclosure.

In other words, the medical questionnaire support system facilitates a patient to answer questions for history taking by himself/herself without depending on a doctor or a nurse. Moreover, with the system, the patient himself/herself can answer minimum necessary question items beforehand on an electronic device terminal. Furthermore, the patient himself/herself can print the answers (the results of history taking) on sticker paper and give the printed paper to, for example, a nurse. Consequently, for example, the nurse can complete a paper medical chart simply by attaching the sticker paper to the chart. Therefore, the medical questionnaire support system can achieve a dramatic increase in efficiency in terms of the time taken for history taking and the time taken to create a medical chart.

An embodiment of the present disclosure is presented below.

[1] A recording medium where a medical questionnaire support program is recorded, the medical questionnaire support program causing an electronic device to execute: a symptom time checking step of checking the time of onset of a problem; a problem checking step of checking an item related to the problem in a clinical department; and a printing step of printing answer results of the steps on printing paper to be affixed.

[2] The recording medium where the medical questionnaire support program is recorded according to [1], wherein the medical questionnaire support program causes the electronic device to further execute an allergy checking step of checking allergies.

[3] The recording medium where the medical questionnaire support program is recorded according to [1] or [2], wherein the medical questionnaire support program causes the electronic device to further execute a smoking frequency checking step of checking frequency of cigarette smoking.

[4] The recording medium where the medical questionnaire support program is recorded according to any one of [1] to [3], wherein the clinical department in the problem checking step is any of ophthalmology, gynecology, pediatrics, cardiovascular medicine, gastroenterology, respiratory medicine, neurology, neuropsychiatry, otolaryngology, dentistry, and dermatology.

[5] The recording medium where the medical questionnaire support program is recorded according to any one of [1] to [3], wherein the clinical department is ophthalmology, and the problem checking step includes: a target eye checking step of checking which eye or eyes have a symptom; an ophthalmic symptom checking step of checking the symptom of a target eye; and a corrective device checking step of checking use or non-use of a corrective device for the eyes.

[6] The recording medium where the medical questionnaire support program is recorded according to [5], wherein the ophthalmic symptom checking step includes selecting one or more items from items: “blurred vision”, “reduced eyesight”, “black floaters in vision”, “double vision”, “for fundus examination”, “eye pain”, “redness”, “itching”, “mucus discharge”, “swollen eyelid”, “tired eyes”, “watering”, “dryness”, “sandy or gritty feeling”, “drooping eyelid”, and “other”.

[7] The recording medium where the medical questionnaire support program is recorded according to [5] or [6], wherein the medical questionnaire support program causes the electronic device to further execute a past medical history checking step of checking a past medical history.

[8] The recording medium where the medical questionnaire support program is recorded according to [7], wherein the past medical history checking step includes: selecting any of items: “no” and “other”; and selecting one or more items from items: “cataracts”, “glaucoma”, “diabetic retinopathy”, “eye floater”, “age-related macular degeneration”, “ pterygium”, “retinal hemorrhage”, “retinal detachment”, “dry eyes”, “allergic conjunctivitis”, and “crossed eyes”.

[9] A medical questionnaire support system including: a personal computer including the recording medium where the medical questionnaire support program is recorded according to any one of [1] to [8]; and a printer. The printer is, for example, a thermal printer.

The embodiment of the present disclosure makes it possible to provide a medical questionnaire support system based on the precondition that paper medical charts are used.

### I. Outline of the Embodiment

A medical questionnaire support program of the embodiment causes an electronic device to execute: a symptom time checking step of checking the time of onset of a problem; a problem checking step of checking an item related to the problem in a clinical department; and a printing step of printing the answer results of the steps on printing paper to be affixed.

Consequently, it is easier for a patient to answer questions for history taking by himself/herself without depending on a doctor or nurse. Moreover, the patient himself/herself can answer minimum necessary question items beforehand on an electronic device terminal. Furthermore, the patient himself/herself can print the answers (the results of history taking) on sticker paper and give the printed paper to, for example, a nurse. In addition, for example, the nurse can complete a paper medical chart simply by attaching the sticker paper to the chart. Therefore, it is possible to achieve a dramatic increase in efficiency in terms of the time taken for history taking and the time taken to create a medical chart.

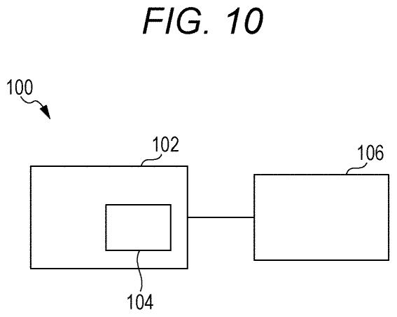

Moreover, as illustrated in FIG. 10, a medical questionnaire support system 100 as another aspect in the embodiment includes a personal computer 102 having the medical questionnaire support program, that is, the personal computer 102 having a storage 104 being a recording medium where the medical questionnaire support program is recorded, and a printer 106. The printer 106 is not particularly limited, but is, for example, a thermal printer.

The recording medium as the storage 104 can be read by the personal computer 102, and is, for example, a non-transitory recording medium that can hold recorded contents even if the supply of power is interrupted. For example, a hard disk drive, a solid state drive, flash ROM, or a detachable USB flash drive can be used as the recording medium. As an example of the electronic device, the personal computer 102 can read the medical questionnaire support program recorded in the storage 104 and execute the medical questionnaire support program.

The medical questionnaire support system 100 of the embodiment can be configured as a system including the personal computer 102 and the printer 106. Hence, the cost of introducing the medical questionnaire support system 100 can be reduced to a relatively low price. Furthermore, the medical questionnaire support system 100 can be configured as a stand-alone system. Hence, also in terms of security, the medical questionnaire support system 100 has a high level of security.

The medical questionnaire support system 100 of the embodiment can be configured, further including a barcode reader. With such a configuration, scanning a barcode on a patient card enables identifying the patient as a re-visit patient and facilitates the display of a questionnaire program targeted for re-visit patients. Therefore, it is possible to obtain the effect of further improving the convenience of the medical questionnaire support system 100 of the embodiment.

The symptom time checking step is the step of checking when the symptom of a patient who has visited a hospital started. As long as the time of onset of the symptom can be checked, it is not necessary to particularly limit the symptom time checking step. In this step, a check can be carried out by various methods.

The symptom time checking step can be typically carried out, following a procedure that presents items related to the time of onset of the symptom and causes the patient to select one or more of the items. Examples of the time of onset of the symptom include “this morning”, “yesterday”, “a few days ago”, “a week ago”, and “more than a week ago”.

The problem checking step is the step of carrying out a minimum necessary problem check in a clinical department where the medical questionnaire support program has been introduced. As long as such a minimum necessary problem check can be carried out, it is not necessary to particularly limit the problem checking step. In this step, a problem check can be carried out, using various items or steps.

Examples of such a problem checking step include the step of checking what symptom(s) the patient has (a symptom checking step), and the step of checking where the symptom is (a site checking step).

In the symptom checking step or the site checking step, it is possible to list more specific symptoms or sites as items in a limited manner depending on the clinical department, and cause the patient to select any of the items.

In other words, specific items are listed in a limited manner to cause the patient to select any of the items in the symptom checking step as in an ophthalmic symptom checking step in ophthalmology, which is described below, or in the site checking step as in a target eye checking step in ophthalmology, which is described below.

If each clinical department has a special condition, a clinical department specific item selection step can be added to the problem checking step. In other words, in the clinical department specific item selection step, it is simply required to set an answer item related to a special condition of the clinical department, and an answer item that the patient himself/herself knows relatively correctly.

Examples of such a clinical department specific item selection step include asking if the patient uses a corrective device such as glasses or contact lenses (a corrective device checking step) in a case of ophthalmology, and asking if the patient is pregnant (a pregnancy checking step) in a case of gynecology.

It is not necessary to particularly limit the clinical department in the problem checking step. Various clinical departments can be applied. Examples of such a clinical department include ophthalmology, gynecology, pediatrics, cardiovascular medicine, gastroenterology, respiratory medicine, neurology, neuropsychiatry, otolaryngology, dentistry, and dermatology.

If the clinical department is ophthalmology in the problem checking step, the problem checking step can include the target eye checking step, the ophthalmic symptom checking step, and the corrective device checking step.

The target eye checking step is the step of checking which eye(s) have the symptom. As long as it is possible to check which eye(s) have the symptom, it is not necessary to particularly limit the target eye checking step. In this step, a check can be carried out by various methods.

The target eye checking step is simply required to be typically carried out, following a procedure that presents items: “left eye” and “right eye”, and causes the patient to select any of them.

The ophthalmic symptom checking step is the step of checking the symptom(s) of the eye(s) of the patient who has visited the hospital. As long as it is possible to check the symptom(s) of the eye(s) of the patient, it is not necessary to particularly limit the ophthalmic symptom checking step. In this step, a check can be carried out by various methods.

In the ophthalmic symptom checking step, a method that lists conceivable symptoms as items, and causes the patient to select any of the items can be typically employed. Examples of such selection items include “blurred vision”, “reduced eyesight”, “black floaters in vision”, “double vision”, “for fundus examination”, “eye pain”, “redness”, “itching”, “mucus discharge”, “swollen eyelid”, “tired eyes”, “watering”, “dryness”, “sandy or gritty feeling”, “drooping eyelid”, and “other”. It may be configured in such a manner as to display one or more of the items and cause the patient to select one or more items.

The corrective device checking step is the step of checking whether or not the patient wears a corrective device for the eyes. As long as it is possible to check the use or non-use of a corrective device, it is not necessary to particularly limit the corrective device checking step. A check can be carried out by various methods.

The corrective device checking step is simply required to be typically carried out, following a procedure that presents items: “glasses” and “contact lenses”, and causes the patient to select any of the items.

In the embodiment, it is preferable to perform a past medical history checking step of checking a past medical history. Consequently, it is possible to preliminarily check a past medical history that the patient knows. Hence, the effect can be obtained which can reduce the time of, or promote the efficiency of, history taking by a doctor or nurse.

In the past medical history checking step, a method that lists conceivable medical conditions as items and causes the patient to select any of the items can be typically employed. Examples of such selection items include “cataracts”, “glaucoma”, “diabetic retinopathy”, “eye floater”, “age-related macular degeneration”, “ pterygium”, “retinal hemorrhage”, “retinal detachment”, “dry eyes”, “allergic conjunctivitis”, and “crossed eyes”. In addition to one or more of these items, an item “other” for other medical conditions, and an item “no” for no medical condition are listed to cause the patient to select any of the items.

In the embodiment, it is preferable to perform an allergy checking step of checking allergies. Consequently, it is possible to preliminarily check allergies that the patient knows. Hence, the effect can be obtained which can reduce the time of, or promote the efficiency of, history taking by a doctor or nurse.

The allergy checking step is simply required to typically list only “yes” and “no” as items for the allergy question. In other words, in terms of allergies, there are various allergies such as allergies to medications and food. The patient may not know his/her allergies correctly. Therefore, it is preferable that, for example, a doctor attempts to correctly grasp the specifics of the patient's allergies through communication with the patient. Hence, it is preferable to simply check whether or not the patient has any allergies in the allergy checking step.

In the embodiment, it is preferable to perform a smoking frequency checking step of checking the frequency of cigarette smoking. In other words, it is known that cigarette smoking brings about vasoconstriction due to nicotine, and affects, for example, glaucoma. Therefore, grasping the frequency of cigarette smoking in advance is especially useful, and has the effect that can reduce the time of, or promote the efficiency of, history taking by a doctor or nurse.

The smoking frequency checking step is simply required to be typically carried out, following a procedure that lists items related to the frequency of cigarette smoking and causes the patient to select any of the items. Examples of the items related to the frequency of cigarette smoking include “1 to 10 cigarettes”, “11 to 20 cigarettes”, and “more than a packet” as the number of cigarettes per day”.

### II. Embodiment

The medical questionnaire support program of the present disclosure is described, taking the drawings as examples. The medical questionnaire support program in the embodiment is tailored to ophthalmology, and is configured as an ophthalmic questionnaire support program. Moreover, the medical questionnaire support program in the embodiment can be recorded in the storage 104 of the personal computer 102 in the medical questionnaire support system 100 illustrated in FIG. 10, and performed by the personal computer 102.

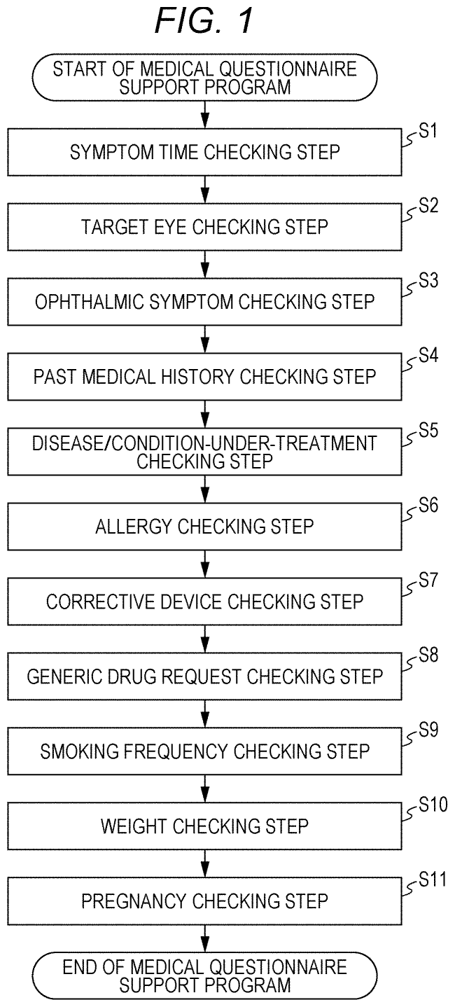

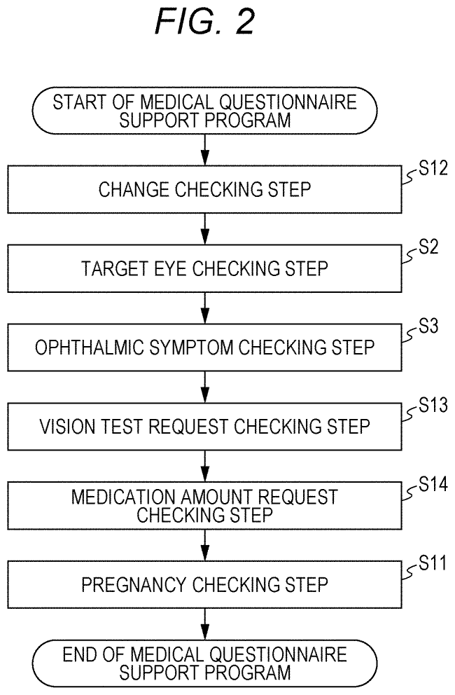

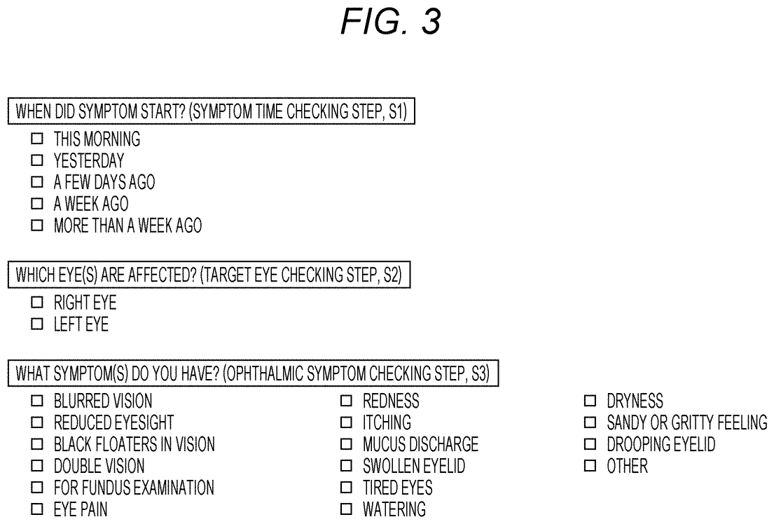

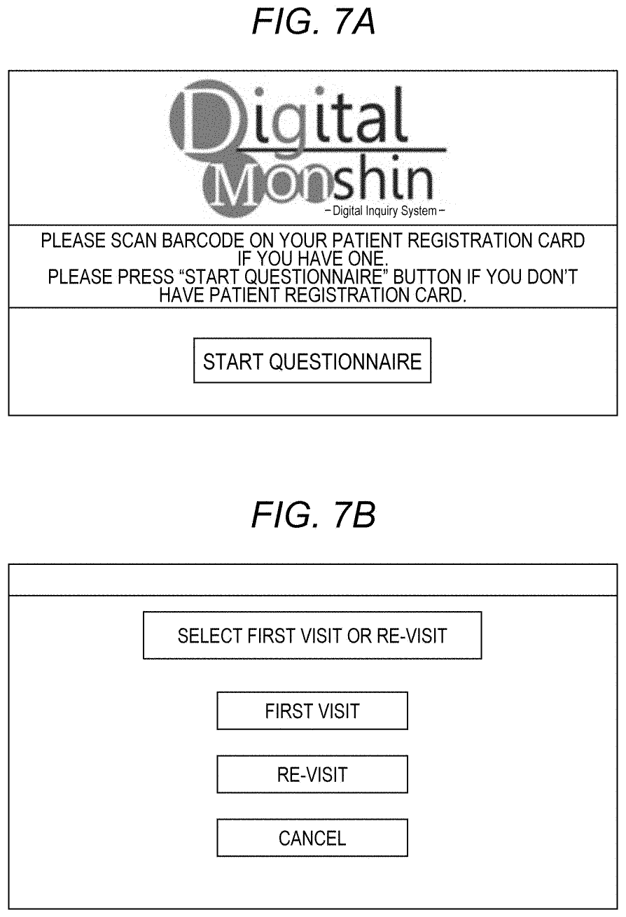

FIG. 1 illustrates an example of a flowchart of the medical questionnaire support program targeted for first visit patients. FIG. 2 illustrates an example of a flowchart of a medical questionnaire support program targeted for re-visit patients. FIGS. 3 to 6 illustrate examples of items displayed. FIGS. 7A to 9G illustrate examples of screens displayed.

If a first visit patient is targeted, various items are checked since the patient visits the hospital for the first time (FIG. 1).

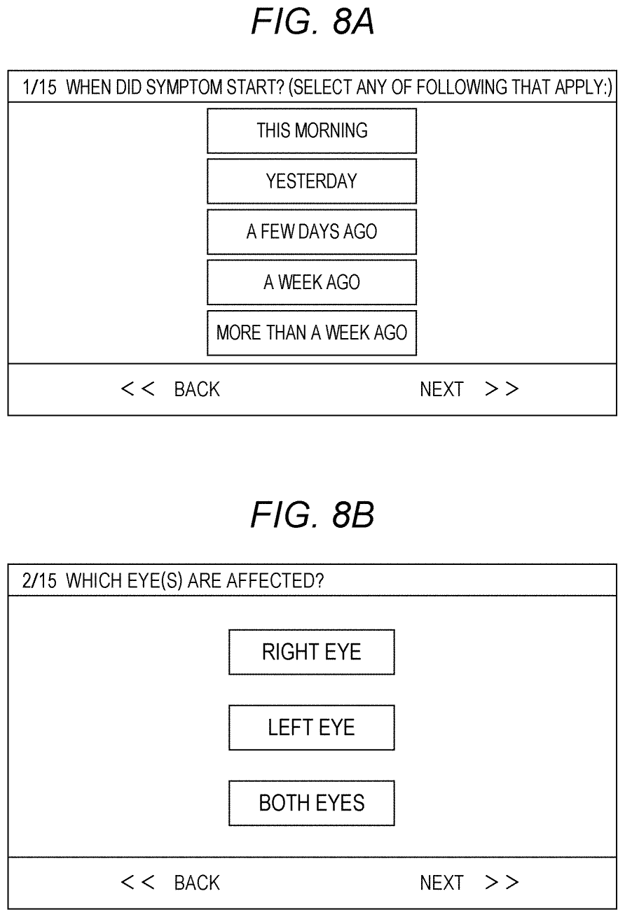

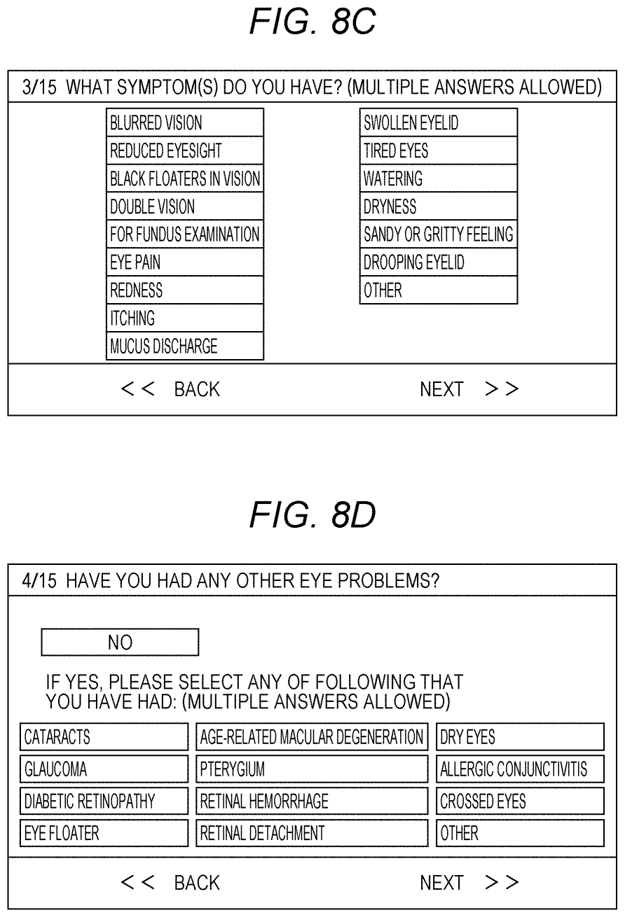

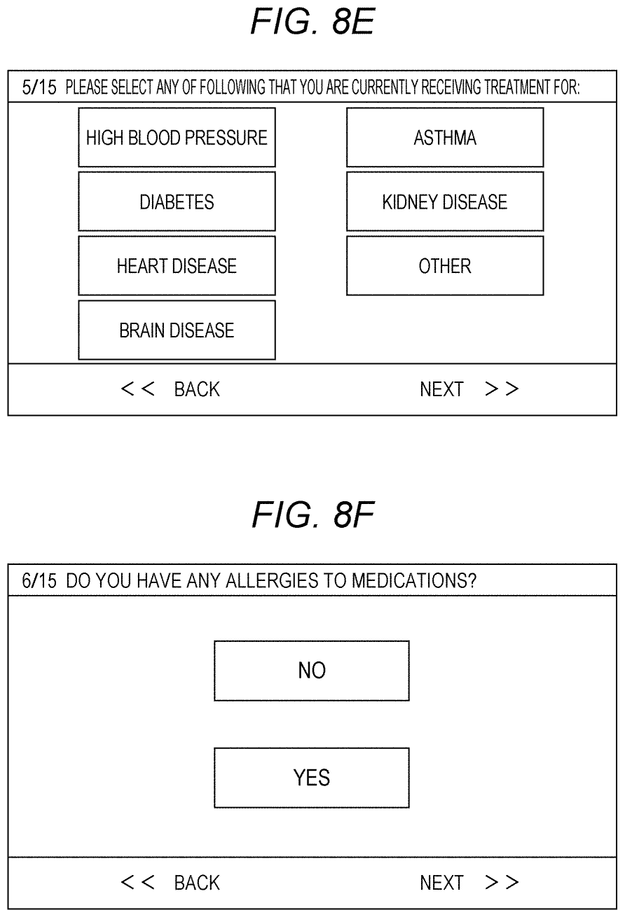

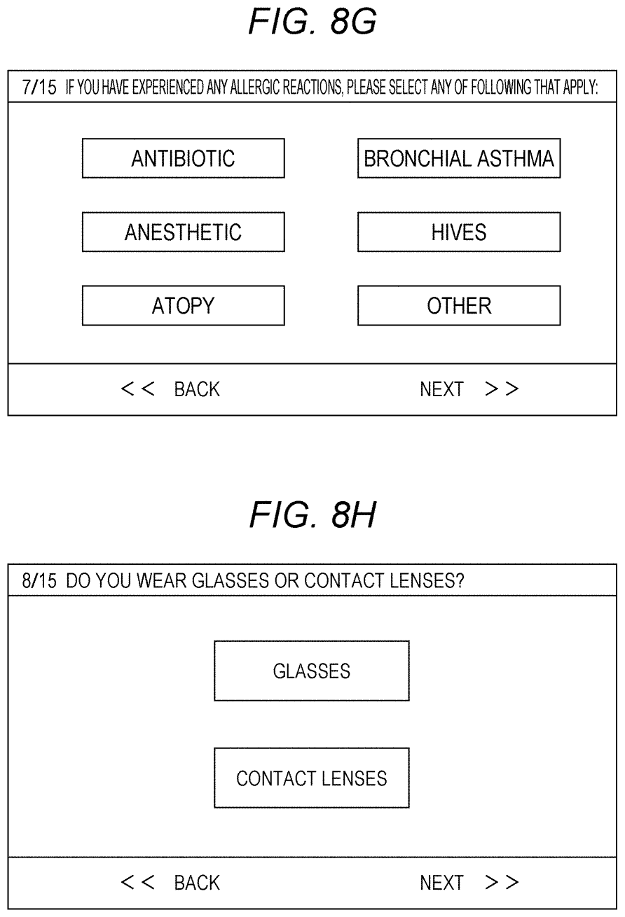

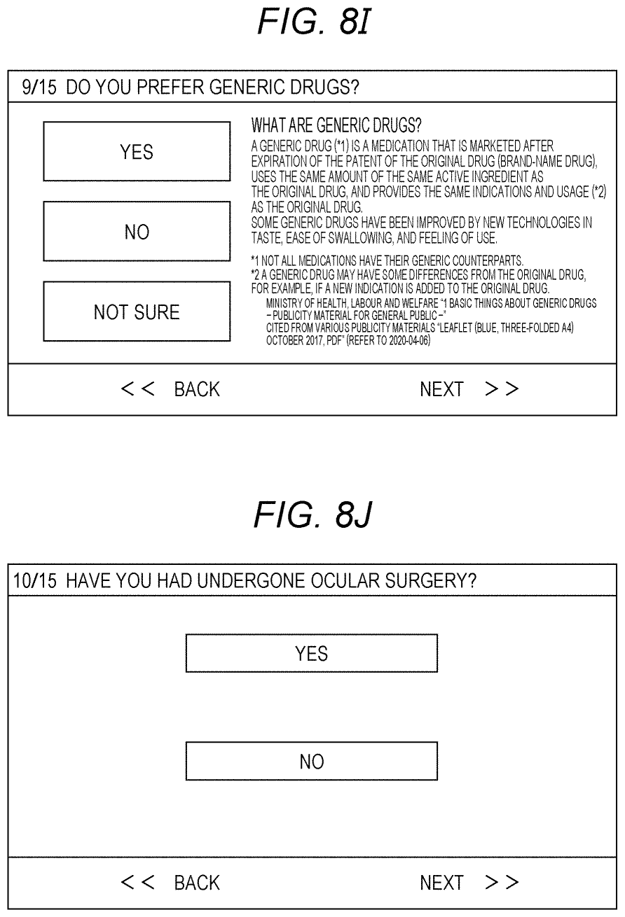

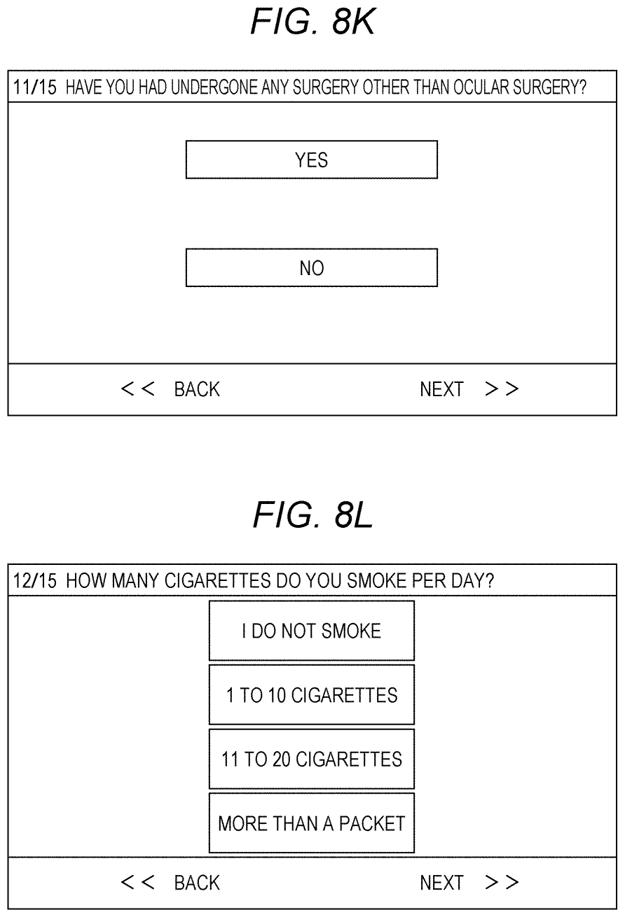

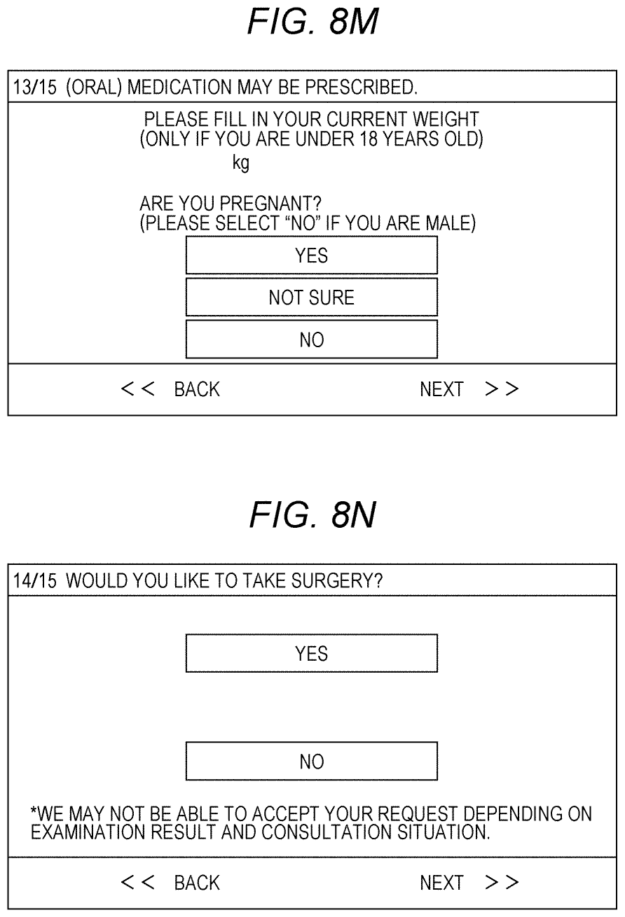

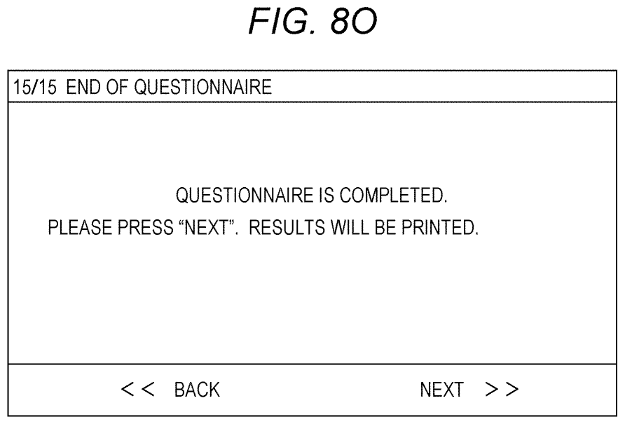

In the symptom checking step S1, a check is carried out, causing the patient to select any of items: “this morning”, “yesterday”, “a few days ago”, “a week ago”, and “more than a week ago” to ask when the symptom started (FIG. 3 and FIG. 8A).

In the target eye checking step S2, a check is carried out, causing the patient to select any of items: “left eye” and “right eye” (FIG. 3 and FIG. 8B).

In the ophthalmic symptom checking step S3, a check is carried out, causing the patient to select any of items: “blurred vision”, “reduced eyesight”, “black floaters in vision”, “double vision”, “for fundus examination”, “eye pain”, “redness”, “itching”, “mucus discharge”, “swollen eyelid”, “tired eyes”, “watering”, “dryness”, “sandy or gritty feeling”, “drooping eyelid”, and “other” (FIG. 3 and FIG. 8C).

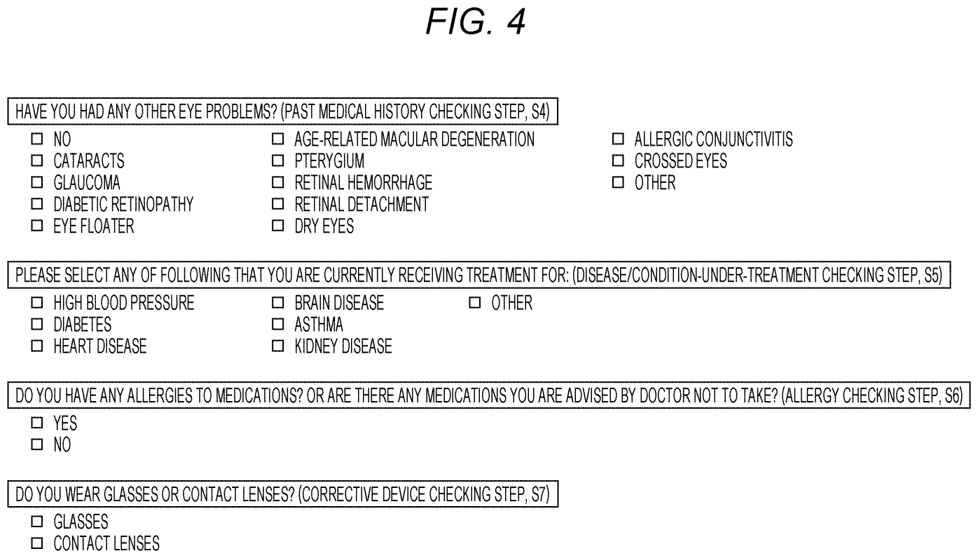

In the past medical history checking step S4, a check is carried out, causing the patient to select any of items: “no”, “cataracts”, “glaucoma”, “diabetic retinopathy”, “eye floater”, “age-related macular degeneration”, “ pterygium”, “retinal hemorrhage”, “retinal detachment”, “dry eyes”, “allergic conjunctivitis”, “crossed eyes”, and “other” (FIG. 4 and FIG. 8D).

In a disease/condition-under-treatment checking step S5, a check is carried out, causing the patient to select any of items: “high blood pressure”, “diabetes”, “heart disease”, “brain disease”, “asthma”, “kidney disease”, and “other” (FIG. 4 and FIG. 8E).

In the allergy checking step S6, a check is carried out, causing the patient to select any of items: “yes” and “no” (FIG. 4 and FIG. 8F). Moreover, if the patient has experienced any allergic reactions, items related to what allergy the patient had are presented to cause the patient to select any of the items (FIG. 8G).

In the corrective device checking step S7, a check is carried out, causing the patient to select any of items: “glasses” and “contact lenses” (FIG. 4 and FIG. 8H).

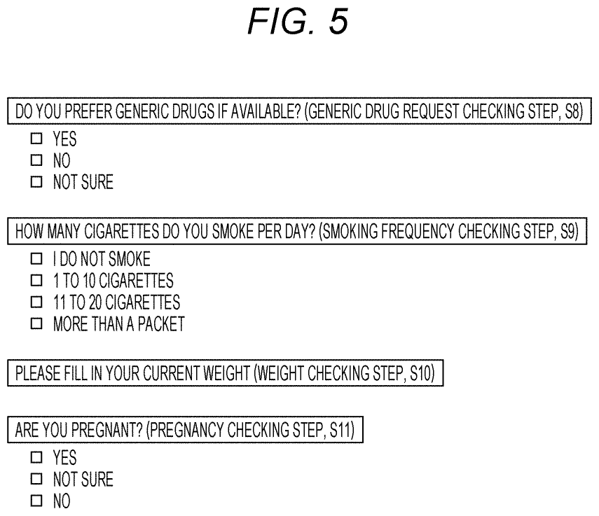

In a generic drug request checking step S8, a check is carried out, causing the patient to select any of items: “yes”, “no”, and “not sure” (FIG. 5 and FIG. 8I).

In the smoking frequency checking step S9, a check is carried out, causing the patient to select any of items: “I do not smoke”, “1 to 10 cigarettes”, “11 to 20 cigarettes”, and “more than a packet” (FIG. 5 and FIG. 8L).

In a weight checking step S10, a check is carried out, causing the patient to fill in the weight in a numerical form (FIG. 5 and FIG. 8M).

In the pregnancy checking step, a check is carried out, causing the patient to select any of items: “yes”, “not sure”, “no” (FIG. 5 and FIG. 8M).

If a re-visit patient is targeted, the hospital has his/her visit history in the recent past, and still keeps the patient's medical record. Therefore, minimum necessary items are checked (FIG. 2). The target eye checking step, the ophthalmic symptom checking step, and the pregnancy checking step are performed as in for first visit.

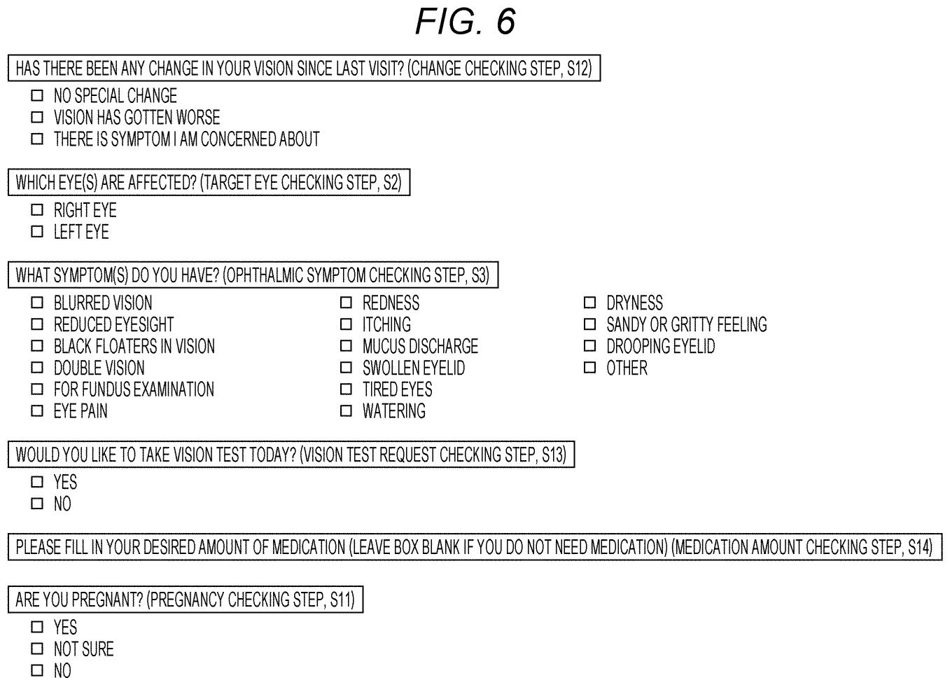

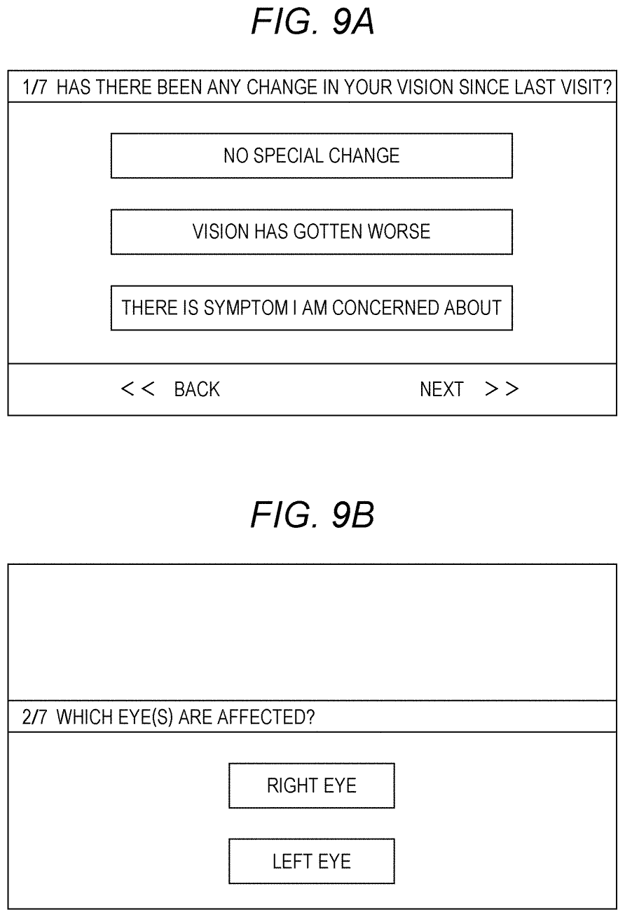

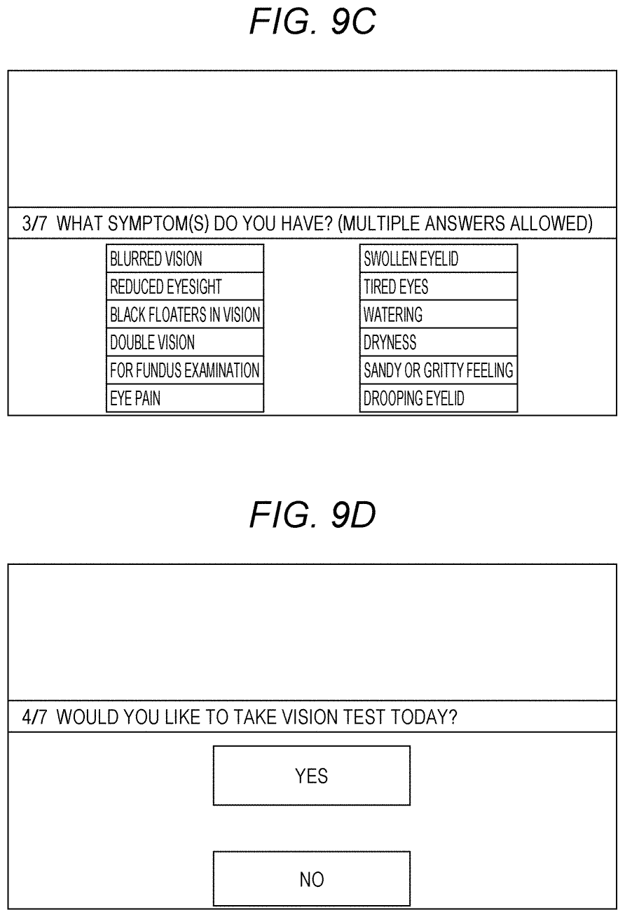

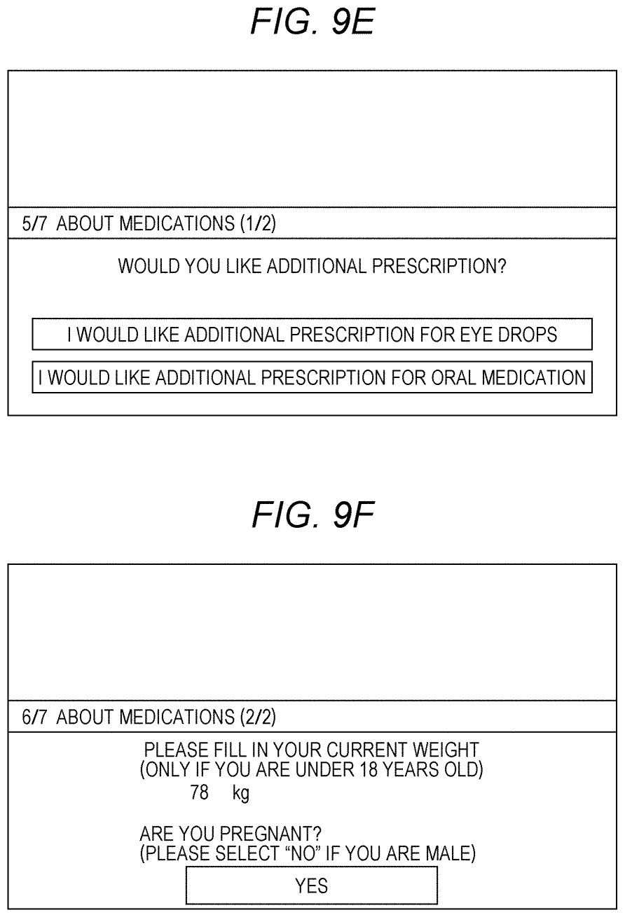

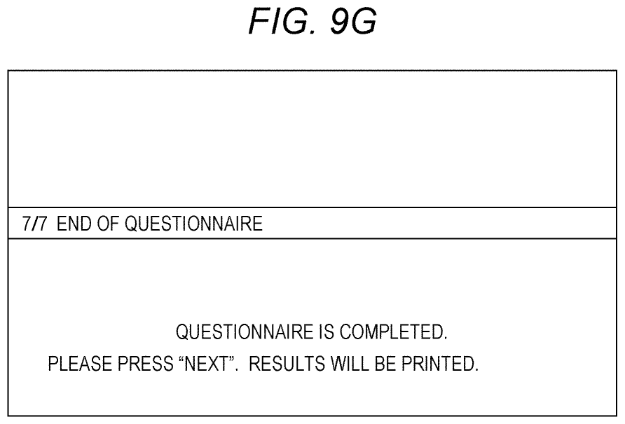

In a change checking step S12, a check is carried out, causing the patient to select any of items: “no special change”, “vision has gotten worse”, “there is a symptom I am concerned about” (FIG. 6 and FIG. 9A).

In a vision test request checking step S13, a check is carried out, causing the patient to select any of items: “yes” and “no” (FIG. 6 and FIG. 9D).

In a medication amount request checking step S14, a check is carried out, causing the patient to fill in whether or not to request a prescription, or the amount of the medication (for xx days) (FIG. 6 and FIG. 9E).

The foregoing detailed description has been presented for the purposes of illustration and description. Many modifications and variations are possible in light of the above teaching. It is not intended to be exhaustive or to limit the subject matter described herein to the precise form disclosed. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims appended hereto.

What is claimed is:

1. A recording medium where a medical questionnaire support program is recorded, the medical questionnaire support program causing an electronic device to execute:
a symptom time checking step of checking the time of onset of a problem;
a problem checking step of checking an item related to the problem in a clinical department; and
a printing step of printing answer results of the steps on printing paper to be affixed.

2. The recording medium where the medical questionnaire support program is recorded according to claim 1, wherein the medical questionnaire support program causes the electronic device to further execute an allergy checking step of checking allergies.

3. The recording medium where the medical questionnaire support program is recorded according to claim 1, wherein the medical questionnaire support program causes the electronic device to further execute a smoking frequency checking step of checking frequency of cigarette smoking.

4. The recording medium where the medical questionnaire support program is recorded according to claim 1, wherein the clinical department in the problem checking step is any of ophthalmology, gynecology, pediatrics, cardiovascular medicine, gastroenterology, respiratory medicine, neurology, neuropsychiatry, otolaryngology, dentistry, and dermatology.

5. The recording medium where the medical questionnaire support program is recorded according to claim 1, wherein
the clinical department is ophthalmology, and
the problem checking step includes:
a target eye checking step of checking which eye or eyes have a symptom;
an ophthalmic symptom checking step of checking the symptom of a target eye; and
a corrective device checking step of checking use or non-use of a corrective device for the eyes.

6. The recording medium where the medical questionnaire support program is recorded according to claim 5, wherein the ophthalmic symptom checking step includes selecting one or more items from items: “blurred vision”, “reduced eyesight”, “black floaters in vision”, “double vision”, “for fundus examination”, “eye pain”, “redness”, “itching”, “mucus discharge”, “swollen eyelid”, “tired eyes”, “watering”, “dryness”, “sandy or gritty feeling”, “drooping eyelid”, and “other”.

7. The recording medium where the medical questionnaire support program is recorded according to claim 5, wherein the medical questionnaire support program causes the electronic device to further execute a past medical history checking step of checking a past medical history.

8. The recording medium where the medical questionnaire support program is recorded according to claim 7, wherein the past medical history checking step includes:
selecting any of items: “no” and “other”; and
selecting one or more items from items: “cataracts”, “glaucoma”, “diabetic retinopathy”, “eye floater”, “age-related macular degeneration”, “ pterygium”, “retinal hemorrhage”, “retinal detachment”, “dry eyes”, “allergic conjunctivitis”, and “crossed eyes”.

9. A medical questionnaire support system comprising:
a personal computer including the recording medium where the medical questionnaire support program is recorded according to claim 1; and
a printer.
